(19) United States
(12) Patent Application Publication
(10) Pub. No.: US 2010/0113631 A1
(43) Pub. Date: May 6, 2010

(54) HALOBENZOATE ESTERS, FLAME RETARDANT COMPOSITION CONTAINING SAME AND, POLYURETHANE FOAM MADE THEREWITH

(76) Inventors: Anantha N. Desikan, Dix Hills, NY (US); Andrew M. Piotrowski, Yorktown Heights, NY (US)

Correspondence Address: DILWORTH & BARRESE, LLP, 1000 WOODBURY ROAD, SUITE 405, WOODBURY, NY, 11797, US

(21) Appl. No.: 12/312,554

(22) PCT Filed: Nov. 19, 2007
(86) PCT No.: PCT/US2007/024119

§ 371 Date: Jan. 20, 2010

Related U.S. Application Data
(60) Provisional application No. 60/859,872, filed on Nov. 17, 2006.

(51) Int. Cl. C09K 21/12; C07C 69/76; C09K 21/08; C08J 9/00

(52) U.S. Cl. 521/107; 560/103; 560/106; 252/601

(57) ABSTRACT

There is provided herein a hydroxyl group-terminated halobenzoate ester of the general formula (1), wherein each X independently is bromine, chlorine, or hydrogen, provided that at least one X is bromine or chlorine, and R is a divalent organic group of from 1 to about 20 carbon atoms, wherein R can optionally contain at least one additional hydroxyl group. There is also provided herein a flame-retardant composition, which comprises a first flame-retardant which is at least one of the aforementioned ester and further comprises a second flame-retardant which is at least one phosphate ester. There is provided flame-retardant polyurethane foams comprising said ester or said composition. There is still also provided herein, process(es) of making the hydroxyl group-terminated halobenzoate ester of the general formula (1) wherein R is a divalent organic group of from 2 to about 20 carbon atoms, wherein R can optionally contain at least one additional hydroxyl group, and polyurethane foams made using the same.

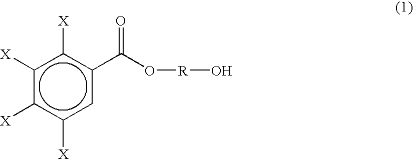

### BACKGROUND OF THE INVENTION

(1) Field of the Invention

The present inventors have unexpectedly discovered a novel halobenzoate ester compound which can be used in a flame-retardant composition and polyurethane foam containing said composition.

(2) Brief Description of Related Art

Polyurethane resins, which are typical of thermosetting resins, are relatively inexpensive and are easy to mold, and the foamed products thereof are widely used over the entire range of articles of daily use, including automotive parts. However, polyurethane resins are flammable. Various efforts have thus been made to produce flame-retardant polyurethane foam. Nowadays, flame retardance is legally compulsory in some fields featuring the use of polyurethane, such as automotive interiors.

Furthermore, flexible polyurethane foam producers are being pressured to produce foam that meets flammability standards with higher fire safety requirements when used in furniture applications. California has proposed increasing the flammability criteria in the form of its new draft of Bulletin 117. The U.S. Congress and the United States Consumer Product Safety Commission are moving toward a national fire safety standard for upholstered furniture.

Flame-retardants based upon pentabromodiphenyl oxide (PBDE) and alkylated triphenyl phosphates have traditionally been used to meet the requirements of the California bulletin 117 as mentioned in U.S. Pat. No. 4,746,682. The market place is seeking alternatives to pentabromodiphenyl oxide.

Generally, in order to impart flame retardancy to polyurethane, the method of adding a halogen-containing phosphate ester as a flame retardant is adopted. Additive-type flame-retardants of halogen-containing monomeric phosphate ester are usually used. However, these flame-retardants tend to create difficulties when used in various applications, such as when they are used in automobile applications. Some vaporization of the flame-retardant from the variations in temperature inside an automobile can occur in some instances, which can cause fogging of interior surfaces of the automobile.

A method, which has been proposed in order to reduce the amount of flame retardant, which can create these difficulties, is to use additive-type flame-retardants of halogen containing condensed phosphate oligomer. However, monomer components generally remain in an amount of about 5 to 20 wt % in oligomer types of flame retardants, so even when oligomer types of flame retardants are used, the problem of the flame retardant becoming vaporized at certain temperatures cannot still be overcome due to the presence of such low molecular weight components.

Methods that have thus been studied include preventing flame-retardants from being vaporized by using flame-retardants with reactive functional groups, referred to as reactive flame-retardants, which are incorporated into the resin skeleton of the polyurethane foam by reacting with starting materials.

The polyurethane foam is formed by the reaction between isocyanate groups of a polyisocyanate and two types of hydroxyl groups, i.e. hydroxyl groups in the polyol and hydroxyl groups in water serving as the blowing agent. However, when reactive flame-retardants of phosphate ester containing reactive functional groups are used, it is necessary to control the reaction between the isocyanate groups and three different types of reactive functional groups, making it difficult to fully satisfy the intended properties of foamed product in the conventional manner.

The direct synthesis of diesters of polyhaloaromatic carboxylic acids and anhydrides for use as flame-retardants when they are incorporated into a variety of inflammable thermosetting and/or thermoplastic polymers has been accomplished in the prior art through esterification of tetrabromobenzoic acid using an expensive metal or organometallic esterification catalyst. See, U.S. Pat. Nos. 5,049,697 and 5,208,366, both to Bohen et al. A disadvantage to this method is that tetrabromobenzoic acid is not readily available, and therefore must be synthesized prior to esterification.

What is needed is a flame retardant system that meets the more stringent flammability requirements and has similar or improved efficiency as compared to the traditional PBDE based flame-retardants, yet because of its reactivity provides permanency. There also remains a need for a reactive flame retardant that better controls the reaction between the isocyanate groups and the three different types of reactive functional groups. Furthermore, there exists a need for a simpler, faster and less expensive method of forming flame-retardants in high yield, preferably reactive flame-retardants.

### BRIEF DESCRIPTION OF THE INVENTION

The inventors have unexpectedly discovered a halogenated benzoate ester that imparts excellent flame-retardancy and minimizes discoloration in polyurethane foam made therefrom.

According to one aspect of the invention herein, there is provided a hydroxyl group-terminated halobenzoate ester of the general formula (1):

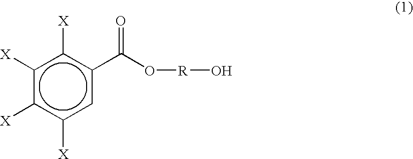

wherein each X independently is bromine, chlorine, or hydrogen, provided that at least one X is bromine or chlorine, and R is a divalent organic group of from 1 to about 20 carbon atoms, wherein R can optionally contain at least one additional hydroxyl group.

According to another aspect of the invention herein, there is provided a flame-retardant composition comprising:

a) a first flame-retardant which is at least one hydroxyl group-terminated halobenzoate ester of the general formula (1):

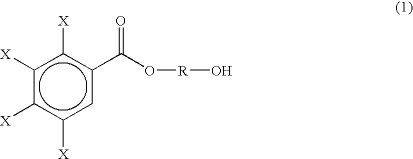

wherein each X independently is bromine, chlorine, or hydrogen, provided that at least one X is bromime or chlorine and R is a divalent organic group of from 1 to about 20 carbon atoms, wherein R can optionally contain at least one additional hydroxyl group; and,

b) a second flame-retardant which is at least one phosphate ester.

In one other aspect of the invention herein there is provided a polyurethane foam made using the hydroxyl group-terminated halobenzoate ester described above.

In one other aspect of the invention herein there is provided a polyurethane foam made using the flame-retardant composition described above.

In yet another aspect of the invention herein there is provided a process of producing a hydroxyl group-terminated halobenzoate ester of the general formula (1):

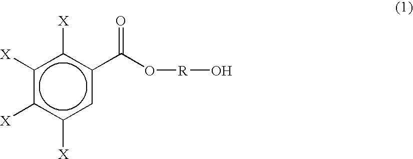

wherein each X independently is bromine, chlorine, or hydrogen, provided that at least one X is bromine or chlorine, and R is a divalent organic group of from 2 to about 20 carbon atoms comprising:

a) reacting halophthalic anhydride of the general formula (2):

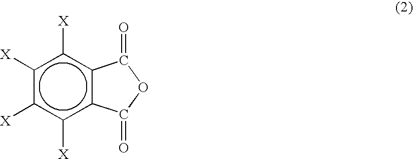

wherein each X independently is bromine, chlorine, or hydrogen, provided that at least one X is bromine or chlorine, with at least one alkanol possessing at least 2 carbon atoms and containing at least two hydroxyl groups; and,

b) decarboxylating substantially any carboxylic acid moieties present in the reaction product of (a) to produce the at least one hydroxyl group-terminated halobenzoate ester of formula (1).

Still even further there are provided polyurethane foam(s) made using any of the hydroxyl-group terminated halobenzoate ester(s) described above, or any of the above-described flame-retardant composition(s).

### DETAILED DESCRIPTION OF THE INVENTION

There is provided herein, a hydroxyl group-terminated halobenzoate ester of formula (1) above, which can be used in a flame retardant composition further comprising at least one phosphate ester, and wherein said hydroxyl group-terminated halobenzoate ester or flame-retardant composition can be used to make resin materials, such as preferably, polyurethane foam, more preferably flame-retardant polyurethane foam. Compared to U.S. Pat. No. 5,637,767, with regard to the process of making benzoate esters, the inventors herein surprisingly have found a novel benzoate ester and a process of making the same, wherein said process uses diols and/or polyols and does not lead to oligomer or polymer formation but to a hydroxyl-terminated benzoate ester which can be used as a flame retardant.

It will be understood herein, that all ranges include all ranges there between and any combination of said ranges or said there between ranges.

It will be understood herein, that all percents' are weight percents' based on the total weight of flame-retardant composition described herein, unless indicated otherwise.

It will be understood herein that that all ranges stated herein comprise all subranges there between, and can further comprise any combination of ranges and/or subranges.

Although each X can independently be bromine, chlorine, or hydrogen, preferably herein each X is bromine so that hydroxyl group-terminated halobenzoate ester of formula (1) is a hydroxyl group-terminated tetrabromobenzoate ester. In one embodiment herein, X is such that at least 3 or 4 X's are each independently bromine or chlorine.

In formula (1), R can be an organic group of up to about 20 carbon atoms, preferably of up to about 12 carbon atoms and more preferably of up to about 8 carbon atoms, and specifically R can optionally contain at least one additional hydroxyl group, and more specifically at least two other hydroxyl groups. In one embodiment, R is divalent, trivalent or tetravalent, preferably divalent. Preferably herein, the organic group can be a hydrocarbon group of up to about 12 carbon atoms, optionally containing at least one heteroatom in said hydrocarbon group. More preferably the organic group can be a divalent hydrocarbon group (alkylene group) of up to about 8 carbon atoms, optionally containing at least one heteroatom in said hydrocarbon group. In one embodiment, R organic group of formula (1) as described herein, can be linear, branched or cyclic, with the herein described ranges of carbon atoms, with the understanding that a branched R group can contain at least 4 carbon atoms and a cyclic R group can contain at least 5 or 6 carbon atoms. Most preferably herein, each X is bromine, and R is a linear, branched or cyclic divalent hydrocarbon group of up to about 8 carbon atoms, optionally containing at least one heteroatom in said hydrocarbon group. Preferably herein, the at least one heteroatom can be any one or more of oxygen, nitrogen, or sulfur, most preferably at least one oxygen. More preferably herein R of formula (1) can be substituted with one or more hydroxyl groups and most preferably with two or more hydroxyl groups in addition to the hydroxyl group indicated in formula (1). In one embodiment R of formula (1) is a an organic group, specifically a hydrocarbon group, more specifically a divalent hydrocarbon group, and most specifically a linear, branched or cyclic divalent alkylene group, of from 1 or 2 carbons up to about 20 carbon atoms, more specifically 1 or 2 carbons up to about 12 carbon atoms and most specifically 1 or 2 carbons up to about 8 carbon atoms. In another embodiment cyclic divalent alkylene group can be substituted or unsubstituted. In yet another embodiment R of formula (1) is a divalent linear, branched or cyclic divalent arylene group, of from 6 or 7 carbons up to about 20 carbon atoms, more specifically 6 or 7 carbons up to about 12 carbon atoms and most specifically 6 or 7 carbons up to about 10 carbon atoms. In yet even another embodiment arylene group can be substituted or unsubstituted.

Preferably herein, the hydroxyl group-terminated halobenzoate ester of formula (1) can comprise where R is a divalent organo group selected from the group consisting of methylene, ethylene, propylene, isopropylene, butylene, isobutylene, pentylene, isopentylene, hexylene, isohexylene, 2-ethylhexylene, cyclohexylene, methylcyclohexylene, butoxyethylene and combinations thereof.

Most preferably herein, hydroxyl group-terminated halobenzoate ester of formula (1) can be selected from the group consisting of hydroxyethyl, hydroxypropyl, hydroxybutyl, cyclohexylmethanol, and combinations thereof.

The flame-retardant composition herein preferably comprises a first flame-retardant composition which is at least one hydroxyl group-terminated halobenzoate ester of formula (1) which is described herein and a second flame-retardant which is at least one phosphate ester, more preferably wherein the phosphate ester is a monomeric and/or oligomeric phosphate ester. In one other embodiment phosphate ester is preferably a halogenated monomeric and/or oligomeric phosphate ester. The flame-retardant composition herein comprises the hydroxyl group-terminated halobenzoate ester of general formula (1) which is described above and a phosphate ester of the general formula (3):

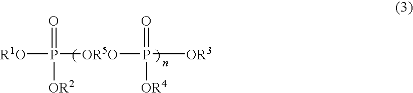

wherein n is 0, or an integer of 1 to 10, wherein, R¹, R², R³, and R⁴ each independently is a non-halogenated or halogenated unsubstituted alkyl group of from 1 to about 10 carbon atoms, a non-halogenated or halogenated substituted alkyl group of from 4 to about 10 carbon atoms, a non-halogenated or halogenated aryl group of from 6 to about 20 carbon atoms, or a non-halogenated or halogenated substituted aryl group of from 7 to about 20 carbon atoms, wherein preferably, substituted alkyl group and/or substituted aryl group is substituted with at least one (preferably 1 to 3) straight or branched alkyl groups of up to about 6 carbon atoms, most specifically butyl; R⁵ is a non-halogenated or halogenated, substituted or unsubstituted alkylene or arylene group, provided, that when n is 1 to about 10, at least one of R¹, R², R³, R⁴ and R⁵ is substituted with at least one halogen atom, R⁵ is preferably a non-halogenated or halogenated unsubstituted alkylene group of from 1 to about 20 carbon atoms, a non-halogenated or halogenated substituted alkylene group of from 2 to about 20 carbon atoms, a non-halogenated or halogenated unsubstituted arylene group of from 6 to about 20 carbon atoms, a non-halogenated or halogenated substituted arylene group of from 7 to about 20 carbon atoms wherein preferably the substituent of said substituted alkylene group and/or said substituted arylene group is an alkyl of from 1 to about 6 carbon atoms, most preferably butyl. More preferably, each of R¹, R², R³ and R⁴ is independently a non-halogenated or halogenated unsubstituted or substituted phenyl group and R⁵ is a non-halogenated or halogenated unsubstituted alkylene group of from 1 to about 8 carbon atoms or a non-halogenated or halogenated alkyl-substituted alkylene group of from 2 to about 8 carbon atoms. In the foregoing phosphate ester (3), which is described, the halogen is preferably chlorine and n is preferably 0, 1, 2 or 3. In one embodiment herein, it will be understood herein that the term substituted refers to substitution other than any halogen substitution that can be present. In one embodiment herein it will be understood that alkyl group as described for formula (3) can be linear, branched or cyclic alkyl.

In a more specific embodiment herein the phosphate ester of general formula (3) can be an alkyl-substituted phenyl phosphate of the general formula (4):

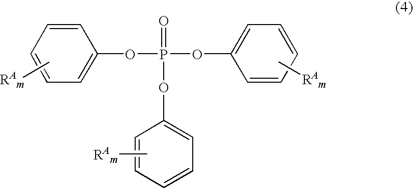

where each R^{A} is independently a linear or branched C₁ to C₆, more specifically, a C₃ to C₆ alkyl group where m is an integer from 1 to 3, but m on at least one phenyl ring is 1 or more.

A preferred embodiment includes R^{A} as a butyl substituent, more preferably i-butyl or t-butyl.

Preferred phosphates that can be used herein) are butylated triphenyl phosphates. It is to be understood that these include pure compounds such as 4-butylphenyl(diphenyl phosphate), as well as mixed triphenyl phosphates in which the individual rings might contain independently 0, 1 or 2 butyl groups, preferably but at least one phenyl ring is butyl substituted. The mixture of alkylated phenyl phosphates is a natural result of the manufacturing process involving alkylated phenols as a starting material. Phenolic starting materials are frequently not uniformly alkylated. Some phenol molecules may not be alkylated at all. Preferably the non-alkylated phenol is not more than 50 wt percent of the phenolic starting material. Economic limitations of phenolic alkylation often make reducing the non-alkylated phenol content below 5 weight percent impractical. More practical is phenol with 20 weight percent non-alkylated. Thus, individual molecules of triphenyl phosphate may have 0, 1, or 2 alkyl substitutions. On average, the level of substitution is more than 0.3, often more than 1, but less than 2. In general, these alkylated triphenyl phosphates will contain in bulk anywhere from about 9 weight percent phosphorus down to about 6.0 weight percent phosphorus depending on the degree of alkylation.

Some preferable examples of the phosphate ester that can be used are monomeric phosphate ester flame retardants such as those selected from the group consisting of tricresyl phosphate, cresyl diphenyl phosphate, propylated triphenyl phosphate, butylated triphenyl phosphate, and combinations thereof.

Some other examples of the phophate ester that can be used herein are monomeric phosphate ester flame retardants such as those selected from the group consisting of tris(1,3-dichloropropyl) phosphate, tris(2,3-dibromopropyl) phosphate, tris(2-chloroethyl) phosphate, and combinations thereof.

In another embodiment herein some preferable examples of oligomeric phosphate ester are selected from the group consisting of Fyrol PNX, Fyrol NDP Fyrol BDP, Fyrol RDP and combinations thereof.

The amount of the hydroxyl group-terminated halobenzoate ester of formula (1) which is present in the flame-retardant composition as described herein is preferably of from about 10 to about 100, more preferably from about 30 to about 70 and most preferably from about 40 to about 60 weight percent, said weight percents being based on the total weight of flame retardant composition. The amount of the phosphate ester, which is present in the flame-retardant composition as described herein, is preferably of from about 0 to about 10, more preferably from about 30 to about 70 and most preferably from about 40 to about 60 weight percent, said weight percents being based on the total weight of flame-retardant composition. In one embodiment the hydroxyl group-terminated halobenzoate ester of formula (1) is present in a blend. In one preferable embodiment the flame-retardant composition can be in the form of a blend wherein the hydroxyl group-terminated halobenzoate ester of formula (1) in a minor amount is blended in the phosphate ester, which is present in a major amount. In another preferable embodiment herein, the flame-retardant composition described herein can be in the form of a blend wherein the phosphate ester in a minor amount is blended in the hydroxyl group-terminated halobenzoate ester of formula (1), which is present in major amount. In one embodiment it is understood herein that a major amount is greater than about 50 weight percent based on the total weight of the flame-retardant composition and a minor amount being less than about 50 weight percent based on the total weight of the flame-retardant composition. In another specific embodiment herein the halobenzoate ester can be blended in the flame-retardant composition in any of the amounts described above. In one embodiment herein hydroxyl group-terminated halobenzoate ester of formula (1), wherein optionally R can contain at least one other hydroxyl group, can be blended in a polyol blend with at least one other polyol, specifically at least two other polyols and more specifically at least 3 other polyols, such as in the situation where the hydroxyl group-terminated halobenzoate ester of formula (1) is solvated in at least one polyol. In yet another embodiment flame-retardant composition herein can be blended in a polyol blend with at least one other polyol, specifically at least two other polyols and more specifically at least 3 other polyols, such as in the situation where the flame-retardant composition is solvated in at least one polyol. In one embodiment there are also provided polyurethane foams, specifically flame-retardant polyurethane foam(s) containing the herein described polyol blend(s) or flame-retardant composition(s) containing said polyol blends, wherein said foams are made using said polyol blends or compositions containing said polyol blends. The flame-retardant composition herein can be used in amounts that flame-retardant compositions are generally used in polyurethane foam-forming mixtures, as is known to those skilled in the art.

In the above-described process of producing the hydroxyl group-terminated halobenzoate ester of formula (1) the at least one halophthalic anhydride of the general formula (2) described above, can comprise wherein each X independently is bromine, chlorine, or hydrogen provided that at least one X is bromine or chlorine, preferably where each X is bromine; and wherein the halophthalic anhydride of the general formula (3) is reacted with at least one alkanol possessing at least two carbon atoms and having at least two hydroxyl groups, preferably at least one diol and/or triol alkanol possessing at least two carbon atoms and having at least two hydroxyl groups, more preferably at least one diol alkanol possessing at least two carbon atoms, and most preferably at least one triol alkanol possessing at least two carbon atoms. More preferably the at least one alkanol possessing at least two carbon atoms and having at least two hydroxyl groups, is of the general formula (5):

R(OH)ₐ (5)

wherein R is a divalent organic group of from 1 to about 20 carbon atoms, preferably from 1 to about 12 carbon atoms, more preferably from 1 to about 8 carbon atoms, and a is an integer of at least two, more preferably a is an integer of from 2 to 8, even more preferably a is an integer of from 2 to 6, and most preferably a is 3. Preferably R of formula (5) is a linear, branched or cyclic organic group, preferably a divalent organic group of up to about 20 carbon atoms, preferably up to about 12 carbon atoms and more preferably up to about 8 carbon atoms. In one embodiment R of formula (5) is an organic group of from 1 to about 20 carbon atoms. More preferably herein, in formula (2), X is bromine and R in formula (5) is a divalent hydrocarbon group of from 2 to about 8 carbon atoms, wherein said hydrocarbon group can be linear, branched or cyclic. Preferably, the alkanol described herein can include alkanols (such as diols in one non-limiting example) having a boiling point of from about 140° C. to about 280° C., more preferably from about 160° C. to about 250° C., and most preferably from about 190° C. to about 230° C. Preferably the alkanol as described above, is a non-halogenated, and/or non-sulfur containing, and/or non-nitrogen-containing alkanol. The term “alkanol” as used herein is understood to include at least one alcohol and/or glycol that possesses at least two carbon atoms and contains at least two hydroxyl groups. In another embodiment herein, it will be understood that R of formula (5) becomes the R of formula (6) and then the R of formula (1) as described herein, when formula (2) is reacted with formula (5) to form formula (6) which is decarboxylated to form formula (1).

Most preferably, the alkanol is selected from the group consisting of 1,2-ethane diol; glycerin; 1,2-propane diol; ethylene glycol; 1,3-propane diol; 1,2-butanediol; 1,3-butanediol; 1,5-pentanediol; neopentylglycol; alkoxydiols such as diethylene glycol; triethylene glycol; trimethylolethane; trimethylolpropane; and, mixtures thereof.

Alternatively, alkanols with boiling points below 140° C. can be used in combination with a higher boiling alkanol. In particular alkanols with boiling points between about 100° C. and 140° C. may be advantageously used in that manner. Preferred are non-halogenated, and/or non-sulfur-containing, and/or non-nitrogen-containing alkanols with boiling points between about 110° C. and 130° C. Branched chain alkanols are most preferred.

In one non-limiting embodiment herein, the flame-retardant composition herein can optionally use alcohol as a solvent in amounts that are known to those skilled in the art.

It will be understood herein that the reaction of halophthalic anhydride of the general formula (2) with alkanol (5) will produce a reaction product such as that of the general formula (6):

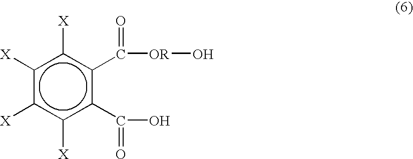

wherein X is as defined above for the general formula (1), and R is an organic group of from 2 to about 20 carbon atoms, and, the carboxylic acid moiety present is subsequently substantially removed to form the hydroxyl group-terminated halobenzoate ester of general formula (1). Removal of the carboxylic acid moiety can be accomplished through processes known to those skilled in the art, preferably removal of the carboxylic acid moiety can be accomplished by a decarboxylation reaction. The decarboxylation reaction can substantially decarboxylate any carboxylic acid moieties present in the reaction product of the halophthalic anhydride (2) and alkanol described herein. Preferably at least about 70 weight percent, of said reaction product is decarboxylated, more preferably at least about 85 weight percent and most preferably at least about 90 weight percent, wherein said weight percents are based on the total weight of the reaction product of halophthalic anhydride of the general formula (2) with the herein described alkanol. In one embodiment the decarboxylation reaction can comprise exposing the reaction product (6) to a temperature greater than about 180° C., more specifically greater than about 190° C. and most specifically greater than about 195° C. but lower than 230° C. to avoid product decomposition. In one non-limiting embodiment herein, decarboxylation can be aided by converting the residual COOH group of formula (6) into a salt such as a Na salt which can reduce the required decarboxylaton temperature; said methods of forming such salts are well known to those skilled in the art.

The decarboxylation reaction can be carried out using any appropriate catalyst that favors decarboxylation over esterification, for example providing in the reaction at hand a reaction product at least 50% by weight comprised of the desired ring-halogenated benzoate compound. Preferred catalysts include alkali carbonates, alkali bicarbonates and caustic alkalis. Sodium and potassium carbonate and bicarbonate, lithium carbonate and sodium aluminate are particularly preferred due to their relatively low cost and ready availability.

The catalyst loading effects the product ratio of halobenzoate of formula (6) to halophthalate ester. With a lower catalyst loading, the decarboxylation step is slower, which allows greater formation of diester. With a higher catalyst loading, lower amounts of diester are formed. The desired catalyst loading is preferably between about 1 and about 25 mole percent, more preferable between about 2 and about 10 mole percent, and most preferably between about 3 and about 6 mole percent based upon the halophthalic anhydride (2) charge.

In one embodiment the reaction of the halophthalic anhydride of formula (2) and the herein described alkanol possessing at least two carbon atoms and having at least two hydroxyl groups (5) can be accomplished with an excess of alkanol, specifically from about 2 to about 7 mole equivalents, more specifically from about 2.5 to about 6.5 mole equivalents and most specifically of from about 3 to about 6 mole equivalents based on the amount of halophthalic anhydride (2) used.

The reaction of the halophthalic anhydride of formula (2) and the herein described alkanol possessing at least two carbon atoms and having at least two hydroxyl groups (5), may be accomplished in an excess of alkanol (e.g., about 3 to about 6 mole equivalents based on the halophthalic anhydride (2) used), although instead of using an excess of alkanol (5), an equivalent amount of alkanol (5) is used and an inert solvent may additionally then be used. One non-limiting example of inert solvent is di(ethyleneglycol) butyl ether or the halobenzoate ester prepared separately. In the later case solvent removal can be avoided since product is used as a solvent.

In one preferred aspect of the invention the reaction of (2) and (5) above, is carried out in inert solvent, such as a high boiling ether, with near stoichiometric amounts of the at least one alkanol (5) (e.g., 1.0 to 1.25 mole equivalents). Inert solvents particularly preferred for the invention have a boiling point of between about 190 degrees Celsius (° C.) and about 260° C. and are preferably from the family of ethers such as, for example, di(ethyleneglycol) dibutyl ether. The inert solvent should have solubility properties, which allow the reaction of the halophthalic anhydride of formula (2) and the herein described alkanol possessing at least two carbon atoms and having at least two hydroxyl groups (5), to proceed at a reasonable rate, particularly by accommodating the solubility of the polar intermediate (or its salt complex).

By this invention, a reaction product can be obtained which contains 50-90% by total weight of the at least one hydroxyl group-terminated halobenzoate ester (preferably hydroxyl group-terminated bromobenzoate ester) and 1-40% by total weight of the corresponding phthalate. In one aspect of the invention a one-pot process provides at least about 60% by total weight halobenzoate, while other preferred aspects provide at least about 70% by total weight halobenzoate. Further preferred aspects provide at least about 80% by total weight halobenzoate, with one aspect of the invention providing at least about 85% by total weight halobenzoate, wherein said total weights are based on the total weight of the reaction product of at least one halophthalic anhydride of formula (2) and the herein described alkanol having at least two hydroxyl groups which is other than 1,4 butane diol (5).

The total organic halogen content (in one non-limiting embodiment, bromine content) of the reaction product of the halophthalic anhydride of formula (2) and the alkanol possessing at least two carbon atoms and having at least two hydroxyl groups, generally falls within the range of about 30 to about 66% % by total weight of the reaction product.

The hydroxyl group-terminated halobenzoate ester produced in accordance with the invention can be used as a flame retardant in a variety of polymer resin systems, in any amount. For example, the hydroxyl group-terminated halobenzoate ester (preferably hydroxyl group-terminated bromobenzoate ester) can be incorporated into thermoset polymers such as polyurethanes by including the hydroxyl group-terminated halobenzoate ester in a polyurethane mixture as the polymer is prepared. Polyurethane mixtures are known to those skilled in the art and will not be described in detail herein. This process has been referred to as the “one-shot” technique, and is described with more particularity in common reference materials such as the Modern Plastics Encyclopedia, Vol. 71, No. 12 (1994). In one preferred embodiment there is provided a polyurethane foam, more preferably a flame-retardant polyurethane foam made using the hydroxyl group-terminated halobenzoate ester described herein preferably, such a polyurethane foam has the hydroxyl group-terminated halobenzoate ester of formula (1) as described herein incorporated into the polyurethane mixture in an amount that allows for an index adjustments and provides foam with desired properties. Polyurethane mixtures and amounts of polyol (specifically formula (1) in one embodiment herein or flame-retardant composition as described herein in another embodiment herein) are well known in the art and will not be described herein. Isocyanates, diisocyanates and the like, and the amounts to use in a polyurethane mixture, are also well known in the art and will not be discussed herein. Preferred examples of isocyanates are toluene-2,4-diisocyanate (TDI) and methylene bisphenyl isocyanate (MDI), which can be used in amounts that are well known in the field of polyurethane chemistry. Preferably herein the resin system is polyurethane, more preferably a polyurethane foam, and can have the flame-retardant composition or the hydroxyl group-terminated halobenzoate ester of formula (1) incorporated in the polyurethane foam (or any resin system) in an amount that will allow the flame-retardant composition to act as a polyol component in a polyurethane mixture. Preferably the flame-retardant composition used in such a polyurethane mixture comprises the hydroxyl group-terminated halobenzoate ester of formula (1) wherein each X is bromine, and R is a linear, branched or cyclic divalent hydrocarbon group of up to about 8 carbon atoms, optionally containing at least one heteroatom, more preferably wherein said heteroatom is at least one oxygen atom. In another preferred embodiment the flame-retardant composition is in the form of a blend as described herein. In yet another preferred embodiment there is provided a polyurethane, preferably a polyurethane foam made using the hydroxyl group-terminated halobenzoate ester (1), as described herein, which hydroxyl group-terminated halobenzoate ester (1) is made by the processes described herein. A most preferred process is wherein in formula (1), each X is bromine, and R is a divalent linear, branched or cyclic hydrocarbon group of up to about 8 carbon atoms. The halobenzoate ester of general formula (1) herein can be used in a polyurethane mixture in an amount that will result in a polyurethane foam with specifically of from about 0.1 weight percent to about 5 weight percent bromine, more specifically of from about 0.2 weight percent to about 4 weight percent bromine and most specifically of from about 0.3 weight percent to about 3.5 weight percent bromine in the polyurethane foam. The composition (flame-retardant composition) herein can be used in a polyurethane foam mixture in an amount that will result in amounts of bromine in polyurethane foam as described above.

The incorporation of the hydroxyl group-terminated halobenzoate ester herein into polyvinyl chloride(s) may also be accomplished by methods known in the art.

It will be understood that other conventional additives may also be incorporated into polymer systems such as that of polyurethane, preferably polyurethane foam described above. For example, the hydroxyl group-terminated halobenzoate ester (1) alone or in combination with the phosphate ester, as described herein, can be incorporated along with other halogenated flame retardant compounds; however, it is preferred in this regard that the hydroxyl group-terminated halobenzoate ester compound (preferably hydroxyl group-terminated tetrabromobenzoate ester (1)) constitute a predominant portion (i.e. greater than about 50 weight percent, more specifically greater than about 60 weight percent) of the total amount of brominated flame retardant included in the system, said weight percent being based on the total weight of brominated flame-retardant in the system. Flame retardant materials such as oxides of Group V elements, especially antimony oxides, and/or phosphorous-containing compounds, can also be included, in addition to, the phosphate ester as described herein. Additional conventional additives which can also be included in composition (flame-retardant composition herein) may include antioxidants, antistatic agents, colorants, fibrous reinforcements, fillers, foaming/blowing agents, catalysts, heat stabilizers, impact modifiers, lubricants, plasticizers, processing aids, UV light stabilizers, crosslinking/curing agents and combinations thereof.

The following examples merely illustrate certain embodiments of the present invention and for that reason should not be construed in a limiting sense.

### EXAMPLES

### Example 1

Tetrabromophthalic anhydride 115.93 g (0.25 moles) was added to 300 ml of 1,3 propanediol preheated to 115° C. This was followed by the addition of 1.8 grams of Na₂CO₃.

The reaction mixture was stirred for 3 hours at 120° C. It was a clear tan solution.

The intermediate product—tetrabromophthalic acid half ester with 1,3 propanediol was formed quantitatively and no residual tetrabromophthalic anhydride was present as indicated by the absence of 171.4, 141.7, 135.9 and 127.1 signals in the ¹³C NMR spectrum.

The reaction mixture was than rapidly heated to 195° C. and maintained at 200-210° C. for 7 hours. At this point the decarboxylation process was completed and final product formed. It was a mixture containing mainly the desired hydroxypropyl ester and tetrabromophthalic acid diester as an impurity (<10%) dissolved in the stating diol.

1,3 propanediol was stripped under vacuum at high temperature 150° C. to about 200° C. by starting the vacuum at 200° C. and allowing the temperature to drop to about 150° C. as a result of solvent evaporation and the residual product (a viscous liquid) was dissolved in toluene, washed with dilute HCl, dilute NaOH and water. Solvent and residual water were removed under reduced pressure. The final product (see structure A below) was characterized by ¹³CNMR in CDCl₃: 31.8 ppm; 59.3 ppm; 63.9 ppm; 123.5 ppm; 125.0 ppm; 131.8 ppm; 132.4 ppm; 132.9 ppm; 135.4 ppm; 165.3 ppm.

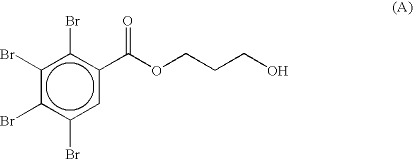

This product was blended with butylated phenyl phosphate ester Phosphlex 71B available from Supresta LLC at 1/1 by weight ratio to form a clear light tan solution.

### Example 2

Tetrabromophthalic anhydride 115.90 g (0.25 moles) was added to 300 ml of anhydrous glycerin preheated to 115° C. This was followed by the addition of 1.7 grams of Na₂CO₃. The reaction mixture was stirred for 3 hours at 120° C. It was a clear colorless solution. The solution was then rapidly heated to 200° C. and maintained at 200-215° C. for 5 hours. A vacuum was than applied and unreacted glycerin was removed. The product was allowed to cool to room temperature. It was a very viscous liquid that crystallized upon storage. It was characterized by ¹³C NMR and was determined to be mainly a dihydroxypropyl tetrabromobenzoate (Formulae B and C below).

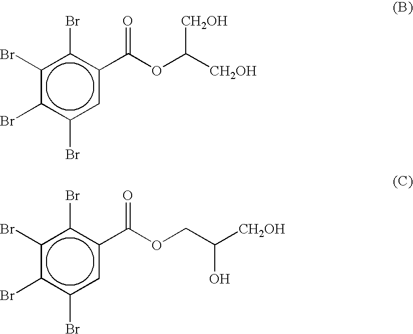

The ¹³C NMR signals which were obtained are listed below:
65.1 ppm; 68.0 ppm; 73.5 ppm; 74.6 ppm; 128.0 ppm; 130.1 ppm; 136.2 ppm; 136.8 ppm; 137.9 ppm; 141.1 ppm; 169.8 ppm.

### Example 3

Flexible polyurethane foams were prepared incorporating a blend as prepared in Example 1 and evaluated for flammability employing test procedures as hereinafter described.

TABLE 1
Flexible Polyurethane Foam Formulations for Example 3

| Component | Example 3 |
|---|---|
| Polyether Polyol VORANOL 3136 from Dow | 100 |
| Chemical | |
| Hydroxypropyl tetrabromobenzoate/ | 18 |
| alkylated triphenylphosphate 50/50 blend | |
| DABCO 33LV/A-1 Amine catalyst available | 0.22 |
| from OSi | |
| Methylene chloride | 10 |
| Water | 5.6 |
| L-620 silicone from OSi | 1 |
| Stannous octoate T-10 from Air Products | 0.56 |
| Toluene diisocyanate (TDI) from Bayer | 71 |
| NCO index | 110 |

Hydroxypropyl tetrabromobenzoate of Table 1 is the product prepared in Example 1. Alkylated triphenylphosphate is Phosphlex 71B available from Supresta LLC.

### Foam Preparation:

Polyol, flame retardant, methylene chloride blowing agent (for 1 pcf density), water, tin and amine catalyst and stabilizer were mixed with stirring in a first beaker. In a separate beaker, the toluene diisocyanate (TDI) was weighed out. A cardboard box for each foam, dimensions of 16″×16″×5.5″, was provided and the catalysts placed in a syringe. The first beaker was stirred at 2100 revolutions per minute for a period of 35 seconds and then the catalysts were dosed thereto while stirring was continued. After a total of 45 seconds of stirring, the TDI was added to the mixture. Stirring was then continued for an additional 7 seconds and the still-fluid mixture was introduced into the box. Once a foam ceased to rise, it was allowed to cure, at first 2-3 hours under a heating lamp and then overnight at room temperature.

### Flammability Evaluation Tests

Flammability was evaluated following STATE OF CALIFORNIA DEPARTMENT OF CONSUMER AFFAIRS BUREAU OF HOME FURNISHINGS AND THERMAL INSULATION TECHNICAL BULLETIN 117 “Requirements, Test Procedure and Apparatus for Testing the Flame Retardance of Resilient Filling Materials Used in Upholstered Furniture” Section A and Section D The results of the Cal TB 117 A Tests are listed below:

TABLE 2
Cal 117 test results

| | Wt. Parts flame | Air | | Cal 117 | Cal 117 |
|---|---|---|---|---|---|
| | retardant/ | flow | Den- | Initial average | Aged average |
| Exam- | 100 parts of | ft³/ | sity, | burn length | Burn length |
| ple | polyol | min | lb/ft³ | inches | inches |
| Exam- | 18 | 3.6 | 1.3 | 3.4″ | 3.4″ |
| ple 1 | | | | | |

What is claimed is:

1. A hydroxyl group-terminated halobenzoate ester of the general formula (1): 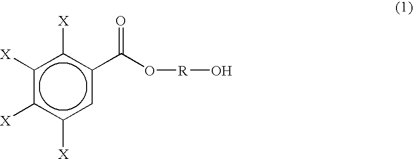
wherein each X independently is bromine, chlorine or hydrogen, provided that at least one X is bromine or chlorine, and R is an organic group of up to about 20 carbon atoms, wherein R can optionally contain at least one additional hydroxyl group.

2. The ester of claim 1 wherein in formula (1), each X is bromine.

3. The ester of claim 1 wherein in formula (1), R is a divalent hydrocarbon group of from 1 to about 12 carbon atoms, optionally containing at least one heteroatom in said hydrocarbon group.

4. The ester of claim 1 wherein in formula (1), R is a linear, branched or cyclic divalent hydrocarbon group of up to about 8 carbon atoms, optionally containing at least one heteroatom in said hydrocarbon group.

5. The ester of claim 1 wherein in formula (1), each X is bromine and R is a linear, branched or cyclic divalent hydrocarbon group of up to about 8 carbon atoms, optionally containing at least one heteroatom in said hydrocarbon group.

6. The ester of claim 1 wherein in formula (1), R is selected from the group consisting of methylene, ethylene, propylene, isopropylene, butylene, isobutylene, pentylene, isopentylene, hexylene, isohexylene, 2-ethylhexylene, cyclohexylene, methyl cyclohexylene, butoxyethylene and combinations thereof.

7. The ester of claim 1 wherein the hydroxyl group-terminated halobenzoate ester is selected from the group consisting of hydroxyethyl, hydroxypropyl, hydroxybutyl, cyclohexylmethanol, and combinations thereof.

8. A polyol blend comprising the ester of claim 1; and, at least one other polyol.

9. A polyol blend comprising the ester of claim 1 wherein said ester contains at least two hydroxyl groups therein; and, at least one other polyol.

10. A flame-retardant composition comprising:
a) a first flame-retardant which is at least one hydroxyl group-terminated halobenzoate ester of the general formula (1): 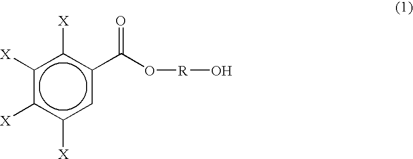
wherein each X independently is bromine, chlorine, or hydrogen, provided that at least one X is bromine or chlorine, and R is an organic group of up to about 20 carbon atoms, wherein R can optionally contain at least one additional hydroxyl group; and,
b) a second flame-retardant which is at least one phosphate ester.

11. The composition of claim 10 wherein phosphate ester (b) is of the general formula (3): 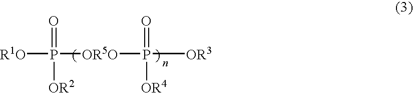
wherein n is 0, or an integer of 1 to 10, wherein, R¹, R², R³, and R⁴ each independently is a non-halogenated or halogenated unsubstituted alkyl group of from 1 to about 10 carbon atoms, a non-halogenated or halogenated substituted alkyl group of from 4 to about 10 carbon atoms, a non-halogenated or halogenated aryl group of from 6 to about 20 carbon atoms, or a non-halogenated or halogenated substituted aryl group of from 7 to about 20 carbon atoms, R⁵ is a non-halogenated or halogenated, substituted or unsubstituted alkylene or arylene group, provided, that when n is 1 to about 10, at least one of R¹, R², R³, R⁴ and R⁵ is substituted with at least one halogen atom.

12. The composition of claim 10 wherein each X is bromine.

13. The composition of claim 10 wherein R is a divalent hydrocarbon group of from 1 to about 12 carbon atoms, optionally containing at least one heteroatom in said hydrocarbon group.

14. The composition of claim 10 wherein R is a linear, branched or cyclic divalent hydrocarbon group of up to about 8 carbon atoms, optionally containing at least one heteroatom in said hydrocarbon group.

15. The composition of claim 10 wherein each X is bromine and R is a linear, branched or cyclic divalent hydrocarbon group of up to about 8 carbon atoms, optionally containing at least one heteroatom in said hydrocarbon group.

16. The composition of claim 10 wherein R is selected from the group consisting of methylene, ethylene, propylene, isopropylene, butylene, isobutylene, pentylene, isopentylene, hexylene, isohexylene, 2-ethylhexylene cyclohexylene, methyl cyclohexylene, butoxyethylene and combinations thereof.

17. The composition of claim 10 wherein the hydroxyl group-terminated halobenzoate ester is selected from the group consisting of hydroxyethyl, hydroxypropyl, hydroxybutyl, cyclohexylmethanol, and combinations thereof.

18. A blend comprising the ester of claim 1.

19. A blend comprising the flame-retardant composition of claim 10, wherein the at least one hydroxyl group-terminated halobenzoate ester is blended in a minor amount of the at least one phosphate ester (b) which is present in a major amount.

20. A polyol blend comprising the flame-retardant composition of claim 10; and at least one other polyol.

21. A flame-retardant polyurethane foam made using the hydroxyl-group terminated halobenzoate ester of claim 1.

22. A flame-retardant polyurethane foam made using the flame-retardant composition of claim 10.

23. A flame-retardant polyurethane foam made using the flame-retardant composition of claim 15.

24. A flame-retardant polyurethane foam made using the blend of claim 18.

25. A flame-retardant polyurethane foam made using the blend of claim 19.

26. A flame-retardant polyurethane foam made using the blend of claim 20.

27. A process of producing the hydroxyl group-terminated halobenzoate ester of the general formula (1): 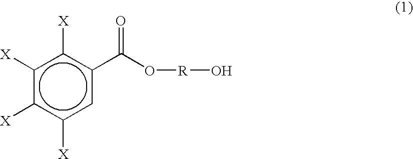
wherein each X independently is bromine, chlorine or hydrogen, provided that at least one X is bromine or chlorine, and R is a divalent organic group of from 2 to about 20 carbon atoms, wherein R can optionally contain at least one additional hydroxyl group; which comprises:
a) reacting halophthalic anhydride of the general formula (2): 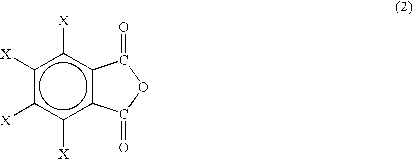
wherein each X independently is bromine, chlorine or hydrogen, provided that at least one X is bromine or chlorine, with at least one alkanol possessing at least two carbon atoms and having at least two hydroxyl groups; and,
b) decarboxylating substantially any carboxylic acid moieties present in the reaction product of (a) to produce the hydroxyl group-terminated halobenzoate ester of formula (1).

28. The process of claim 27 wherein the alkanol is a diol.

29. The process of claim 27 wherein the alkanol is of the general formula (5):
R(OH)ₐ (5)
wherein R is an organic group of from 2 to about 20 carbon atoms and a is an integer of at least two.

30. The process of claim 29 wherein R is a divalent hydrocarbon group of from 2 to about 12 carbon atoms.

31. The process of claim 29 wherein R is a divalent linear, branched or cyclic hydrocarbon group of up to about 8 carbon atoms.

32. The process of claim 29 wherein X is bromine and R is a divalent linear, branched or cyclic hydrocarbon group of up to about 8 carbon atoms.

33. The process of claim 27 wherein the alkanol is selected from the group consisting of 1,2-ethane diol; glycerin; 1,2-propane diol; ethylene glycol; 1,3-propane diol; 1,2-butanediol; 1,3-butanediol; 1,5-pentanediol; neopentylglycol; alkoxydiols such as diethylene glycol; triethylene glycol; trimethylolethane; trimethylolpropane; and, mixtures thereof.

34. The process of claim 27 wherein each X is bromine.

35. A flame-retardant polyurethane foam made using the hydroxyl-group terminated halobenzoate ester made by the process of claim 27.

36. A flame-retardant polyurethane foam made using the hydroxyl-group terminated halobenzoate ester made by the process of claim 32.
